(19) United States
(12) Patent Application Publication
(10) Pub. No.: US 2007/0059360 A1
(43) Pub. Date: Mar. 15, 2007

(54) WATER-DISPERSIBLE ANTI-RETROVIRAL PHARMACEUTICAL COMPOSITIONS

(76) Inventors: Ashish Jaiswal, Indore (IN); Mukesh Garg, Jagroan (IN); Ajay Kumar Singla, Chandigarh (IN)

Correspondence Address: RANBAXY INC., 600 COLLEGE ROAD EAST, SUITE 2100, PRINCETON, NJ, 08540, US

(21) Appl. No.: 11/461,057

(22) Filed: Jul. 31, 2006

(30) Foreign Application Priority Data
Jul. 29, 2005 (IN) 2019/DEL/2005

(51) Int. Cl. A61K 31/551; A61K 31/522; A61K 9/20

(52) U.S. Cl. 424/464; 514/220; 514/263.31

(57) ABSTRACT

Provided herein are water-dispersible pharmaceutical compositions comprising a combination of one or more anti-retroviral drugs useful for the treatment of Human Immunodeficiency Virus (HIV) infections. Also provided are processes for preparing such water-dispersible pharmaceutical compositions.

### FIELD OF THE INVENTION

Provided herein are water-dispersible pharmaceutical compositions comprising a combination of one or more anti-retroviral drugs. Such anti-retroviral drugs can be useful for the treatment of Human Immunodeficiency Virus (HIV) infections. Also provided are processes for preparing such water-dispersible pharmaceutical compositions.

### BACKGROUND OF THE INVENTION

Human immunodeficiency virus (HIV) has been implicated as the primary cause of the slowly degenerate disease of the immune system termed acquired immune deficiency syndrome (AIDS). AIDS predisposes subjects to fatal opportunistic infections. Characteristically, AIDS is associated with a progressive depletion of T-cells, especially the helper-inducer subset bearing the CD4 surface marker. HIV is cytopathic and appears to preferentially infect and destroy T-cells bearing the CD4 marker.

Currently several anti-retroviral drugs are available, which inhibit the growth and replication of HIV at various stages of its life cycle, such as reverse transcriptase inhibitors (RTI's), protease inhibitors and fusion inhibitors. RTI's inhibit the reverse transcriptase enzyme. This enzyme synthesizes double-stranded DNA from the retrovirus own single-stranded RNA genome. This DNA double helix integrates into the host cell's chromosomes as a provirus. Transcription then leads to copies of the viral RNA genome, from which the virus's own proteins and enzymes are formed. New viral particles then bud from the membrane of the cell. Thus, reverse transcriptase inhibition is essential for inhibiting viral replication. RTI's fall into two categories: nucleoside/nucleotide analogs, such as lamivudine, stavudine, zidovudine, didanosine, abacavir, tenofovir, emtricitabine and zalcitabine; and non-nucleoside analogs, such as nevirapine, delavirdine and efavirenz.

U.S. Pat. No. 5,047,407 discloses lamivudine and its use in the treatment and prophylaxis of viral infections.

Nevirapine is a known agent for the treatment of HIV infection and its synthesis and use are described in various publications including, inter alia, U.S. Pat. No. 5,366,972 and European Patent Application No. 429,987.

Stavudine or 2′,3′-didehydro-3′-deoxythymidine (d4T) is a potent inhibitor of HIV reverse transcriptase in vitro reported by S. A. Riddler, et al in Antiviral Research, (1995) 27, 189-203.

Development of drug resistance has recently become a major concern in the treatment of HIV infections. Drug resistance usually occurs when the drugs being used are not potent enough to completely halt virus replication. Moreover, the HIV virus is able to mutate or change frequently and develop resistance to drugs, making HIV infection and AIDS very difficult to treat. Once a mutation occurs, it then grows unchecked and soon becomes dominant strain of the virus in the individual. The drug becomes progressively weaker against the new strain.

Several studies have shown that combining two drugs delays the development of resistance to one or both drugs compared to the use of either drug alone. Combination therapy may help prevent drug failure caused by viral resistance and may decrease the amount of free virus that can infect other cells. Some available agents appear to be beneficial only in combination therapy regimens. Most combinations comprise two anti-retroviral drugs, such as a combination of a protease inhibitor and a RTI, or a combination of nucleoside RTI and a non-nucleoside RTI. It has also been found that therapy using combinations of three or more drugs, such as a protease inhibitor or a non-nucleoside RTI with two nucleoside RTI, may greatly reduce disease progression and deaths in people with AIDS. Such a therapy is also known as HAART (Highly Active Anti-Retroviral Therapy).

However, the success of combination therapy for HIV often requires strict compliance with a complex treatment regimen that can require the administration of many different drugs per day and administered at precisely timed intervals with careful attention to diet. Such complex treatment regimens may lead to potential compliance problems related to non-adherence to therapy. For example, patient non-compliance may be a serious problem in the treatment of HIV because it may lead to the emergence of multiple-drug resistant strains of HIV. An effort to simplify treatment regimens for HIV with the goal of enhancing patient compliance by providing a simplified dosage form was made by designing Fixed-Dose Combinations (FDC's), which combine two, or more active drugs in one tablet or capsule.

PCT Application No. WO 98/18477 discloses solid dosage forms, such as tablets and capsules of a combination of lamivudine and zidovudine with pharmaceutical glidants, which prevent segregation of drugs leading to a homogeneous mixture, thus increasing efficacy.

South African Application No. 2001/10499 discloses pharmaceutical compositions of a bi-layered tablet having a combination of lamivudine, stavudine and nevirapine, or pharmaceutically acceptable derivatives thereof, and a method of preparing such pharmaceutical compositions.

South African Application No. 2001/10501 discloses pharmaceutical compositions of a bi-layered or a conventional tablet having a combination of lamivudine and stavudine or pharmaceutically acceptable derivatives thereof, and a method of preparing such compositions.

All compositions as described above are solid dosage forms either in the form of tablet or capsule. Many patients may have difficulty in swallowing such solid dosage forms, and consequently may not comply with taking medications as prescribed, particularly for pediatric and geriatric patients. This may result in a high incidence of non-compliance and ineffective therapy, which may prove to be fatal in case of a progressive condition, such as AIDS.

Thus, there is a need for oral dosage forms comprising one or more anti-retroviral drugs, which can be taken orally without the need of swallowing a solid dosage form. Accordingly, provided are compositions, which readily disperse in water or another suitable vehicle of administration and can be taken orally.

### SUMMARY OF THE INVENTION

In one aspect, provided herein are water-dispersible pharmaceutical compositions for oral administration comprising one or more anti-retroviral drugs and one or more pharmaceutically acceptable excipients selected from one or more diluents, disintegrants, binders, lubricants, glidants or mixtures thereof.

The pharmaceutical compositions can include one or more of the following embodiments. For example in one embodiment, the water-dispersible pharmaceutical compositions can comprise a combination of at least two anti-retroviral drugs. In one embodiment, the composition comprises:
a) an intragranular portion comprising pharmaceutically acceptable excipients selected from one or more diluents, disintegrants, binders, lubricants, glidants or mixtures thereof and one or more anti-retroviral drugs; and
b) an extragranular portion comprising pharmaceutically acceptable excipients selected from one or more diluents, disintegrants, lubricants, glidants or mixtures thereof and one or more anti-retroviral drugs.

In another embodiment, the composition comprises:
a) an intragranular portion comprising pharmaceutically acceptable excipients selected from one or more diluents, disintegrants, binders, lubricants, glidants or mixtures thereof and one or more anti-retroviral drugs; and
b) an extragranular portion comprising pharmaceutically acceptable excipients selected from one or more diluents, disintegrants, lubricants, glidants or mixtures thereof.

In yet another embodiment, the composition comprises:
a) an intragranular portion comprising pharmaceutically acceptable excipients selected from one or more diluents, disintegrants, binders, lubricants, glidants or mixtures thereof; and
b) an extragranular portion comprising pharmaceutically acceptable excipients selected from one or more diluents, disintegrants, lubricants, glidants or mixtures thereof.

Suitable anti-retroviral drugs are selected from lamivudine, zidovudine, stavudine, abacavir, adefovir, tenofovir, emtricitabine, zalcitabine, didanosine, efavirenz, nevirapine, delavirdine, indinavir, nelfinavir, lopinavir, ritonavir, saquinavir, amprenavir, atazanavir, tipranavir, fosamprenavir or mixtures thereof.

In one embodiment, the pharmaceutical composition disintegrates and disperses in one or more solvents or a vehicle of administration in less than one minute.

Suitable disintegrants are selected from sodium starch glycolate, cross-linked carboxymethylcellulose and its sodium salt, cross-linked polyvinylpyrrolidone, pregelatinised starch, sodium carboxymethyl cellulose, calcium carboxymethyl cellulose, low-substituted hydroxypropyl cellulose, alginates or its salts or mixtures thereof.

Suitable diluents are selected from lactose, dextrose, sucrose, fructose, maltose, powdered cellulose, microcrystalline cellulose, mannitol, erythritol, sorbitol, xylitol lactitol, dicalcium phosphate, tribasic calcium phosphate, calcium sulphate, calcium carbonate or mixtures thereof.

Suitable binders are selected from corn starch, pregelatinised starch, polyvinylpyrrolidone, hydroxypropyl cellulose, hydroxypropyl methylcellulose, carboxyvinyl polymers, acrylates or mixtures thereof.

Suitable lubricants are selected from talc, magnesium stearate, zinc stearate, calcium stearate, sodium stearyl fumarate, stearic acid or mixtures thereof.

Suitable glidants are selected from talc, colloidal silicon dioxide or mixtures thereof.

In one embodiment, the pharmaceutical composition is a tablet.

In another aspect, provided are processes for preparing a water-dispersible pharmaceutical composition for oral administration comprising the steps of:

a. forming a first blend comprising one or more pharmaceutically acceptable excipients selected from one or more diluents, disintegrants, binders, lubricants, glidants or mixtures thereof and one or more anti-retroviral drugs;

b. granulating the first blend by wet or dry granulation to form granules;

c. blending the granules with of one or more pharmaceutically acceptable excipients selected from one or more diluents, disintegrants, binders, lubricants, glidants or mixtures thereof and one or more anti-retroviral drugs to form a second blend; and

d. forming the second blend into a pharmaceutical composition,

In yet another aspect, provided are processes for preparing a water-dispersible pharmaceutical composition for oral administration comprising the steps of:

a. forming a first blend comprising one or more pharmaceutically acceptable excipients selected from one or more diluents, disintegrants, binders, lubricants, glidants or mixtures thereof and one or more anti-retroviral drugs;

b. granulating the first blend by wet or dry granulation to form granules;

c. blending the granules with of one or more pharmaceutically acceptable excipients selected from one or more diluents, disintegrants, binders, lubricants, glidants or mixtures thereof to form a second blend; and

d. forming the second blend into a pharmaceutical composition.

In yet another aspect, provided are processes for preparing a water-dispersible pharmaceutical composition for oral administration comprising the steps of:

a. forming a first blend comprising one or more pharmaceutically acceptable excipients selected from one or more diluents, disintegrants, binders, lubricants, glidants or mixtures thereof;

b. granulating the first blend by wet or dry granulation to form granules;

c. blending the granules with of one or more pharmaceutically acceptable excipients selected from one or more diluents, disintegrants, binders, lubricants, glidants or mixtures and one or more anti-retroviral drugs thereof to form a second blend; and

d. forming the second blend into a pharmaceutical composition.

### DETAILED DESCRIPTION OF THE INVENTION

Provided herein are water-dispersible pharmaceutical compositions for oral administration comprising one or more anti-retroviral drugs and one or more pharmaceutically acceptable excipients selected from one or more diluents, disintegrants, binders, lubricants, glidants or mixtures thereof. In one embodiment, the pharmaceutical compositions comprise at least two anti-retroviral drugs.

Suitable anti-retroviral drugs for use in the water-dispersible pharmaceutical compositions disclosed herein include, but are not limited to, lamivudine, zidovudine, stavudine, abacavir, adefovir, tenofovir, emtricitabine, zalcitabine, didanosine, efavirenz, nevirapine, delavirdine, indinavir, nelfinavir, lopinavir, ritonavir, saquinavir, amprenavir, atazanavir, tipranavir, fosamprenavir or mixtures thereof. Preferred anti-retroviral drugs include lamivudine, stavudine, nevirapine or mixtures thereof.

The water-dispersible pharmaceutical compositions can comprise an intragranular portion and an extragranular portion. The intragranular portion and extragranular portion each comprises one or more pharmaceutically acceptable excipients selected from one or more diluents, disintegrants, binders, lubricants, glidants or mixtures thereof. At least one or both of the intragranular portion and extragranular portion comprise one or more anti-retroviral drug. Thus, water-dispersible pharmaceutical compositions include:

a) an intragranular portion having one or more anti-retroviral drugs and one or more pharmaceutically acceptable excipients selected from one or more diluents, disintegrants, binders, lubricants, glidants or mixtures thereof and an extragranular portion having one or more anti-retroviral drugs and one or more pharmaceutically acceptable excipients selected from one or more diluents, disintegrants, binders, lubricants, glidants or mixtures thereof;

b) an intragranular portion having one or more pharmaceutically acceptable excipients selected from one or more diluents, disintegrants, binders, lubricants, glidants or mixtures thereof and an extragranular portion having one or more anti-retroviral drugs and one or more pharmaceutically acceptable excipients selected from one or more diluents, disintegrants, binders, lubricants, glidants or mixtures thereof; or

c) an intragranular portion having one or more anti-retroviral drugs and one or more pharmaceutically acceptable excipients selected from one or more diluents, disintegrants, binders, lubricants, glidants or mixtures thereof and an extragranular portion having one or more pharmaceutically acceptable excipients selected from one or more diluents, disintegrants, binders, lubricants, glidants or mixtures thereof,

wherein the one or more anti-retroviral drugs are selected from lamivudine, zidovudine, stavudine, abacavir, adefovir, tenofovir, emtricitabine, zalcitabine, didanosine, efavirenz, nevirapine, delavirdine, indinavir, nelfinavir, lopinavir, ritonavir, saquinavir, amprenavir, atazanavir, tipranavir, fosamprenavir or mixtures thereof. Preferred anti-retroviral drugs in such water-dispersible compositions include lamivudine, stavudine, nevirapine or mixtures thereof.

In some embodiments, the intragranular portion can comprise a first granular portion, a second granular portion, and optionally additional granular portions. Each of the first granular portion, second granular portion and optional additional granular portions comprise one or more pharmaceutically acceptable excipients selected from one or more diluents, disintegrants, binders, lubricants, glidants or mixtures thereof. At least one, two or more of the first granular portion, second granular portion and optional additional granular portions comprise one or more anti-retroviral drug. Thus, for example, water-dispersible pharmaceutical compositions described herein can comprise an intragranular portion having:

a) a first granular portion comprising one or more anti-retroviral drugs and one or more pharmaceutically acceptable excipients selected from one or more diluents, disintegrants, binders, lubricants, glidants or mixtures thereof, and a second granular portion comprising one or more anti-retroviral drugs and one or more pharmaceutically acceptable excipients selected from one or more diluents, disintegrants, binders, lubricants, glidants or mixtures thereof; or

b) a first granular portion comprising one or more anti-retroviral drugs and one or more pharmaceutically acceptable excipients selected from one or more diluents, disintegrants, binders, lubricants, glidants or mixtures thereof, and a second granular portion comprising one or more pharmaceutically acceptable excipients selected from one or more diluents, disintegrants, binders, lubricants, glidants or mixtures thereof,

wherein the one or more anti-retroviral drugs are selected from lamivudine, zidovudine, stavudine, abacavir, adefovir, tenofovir, emtricitabine, zalcitabine, didanosine, efavirenz, nevirapine, delavirdine, indinavir, nelfinavir, lopinavir, ritonavir, saquinavir, amprenavir, atazanavir, tipranavir, fosamprenavir or mixtures thereof. Preferred anti-retroviral drugs in such water-dispersible compositions include lamivudine, stavudine, nevirapine or mixtures thereof.

The term “water dispersible,” as used herein, is intended to describe compositions that disintegrate and readily disperse in water, other solvent or a suitable vehicle of administration in less than one minute.

The term “anti-retroviral drugs,” as used herein includes drugs or compounds intended for treating, reversing, reducing or inhibiting retroviral infections, in particular infections caused by HIV. The anti-retroviral drug may be selected from various classes of drugs, such as nucleoside or non-nucleoside reverse transcriptase inhibitors or protease inhibitors. Nucleoside reverse transcriptase inhibitors may include lamivudine, zidovudine, stavudine, abacavir, adefovir, tenofovir, emtricitabine, zalcitabine and didanosine. Non-nucleoside reverse transcriptase inhibitors may include efavirenz, nevirapine and delavirdine. Protease inhibitors may include indinavir, nelfinavir, lopinavir, ritonavir, saquinavir, amprenavir, atazanavir, tipranavir and fosamprenavir. Anti-retroviral drugs includes free base, as well as pharmaceutically acceptable salts, solvates, enantiomers, esters or polymorphs thereof or any compound, which upon administration to the recipient, is capable of providing the anti-retroviral drug or any active metabolite or residue thereof, either directly or indirectly. In some examples, lamivudine may be present in an amount ranging from about 1% to about 20% by weight of the composition. In other examples, stavudine may be present in an amount ranging from about 0.5% to about 10% by weight of the composition. In yet other examples, nevirapine may be present in an amount ranging from about 5% to about 30% by weight of the composition.

Examples of water-dispersible pharmaceutical compositions described herein include, but are not limited to, the following. For example, a water-dispersible pharmaceutical composition for oral administration can comprise:
a) an intragranular portion comprising at least one anti-retroviral drug and one or more of a diluent, disintegrant, binder, lubricant and glidant; and
b) an extragranular portion comprising one or more of a diluent, disintegrant, binder, lubricant and glidant.

In another example, a water-dispersible pharmaceutical composition for oral administration can comprise:
a) a first granular portion comprising at least one anti-retroviral drug and one or more of a diluent, disintegrant, binder, lubricant and glidant;
b) a second granular portion comprising at least one anti-retroviral drug and one or more of a diluent, disintegrant, binder, lubricant and glidant
c) an extragranular portion comprising one or more of a diluent, disintegrant, binder, lubricant and glidant.

In another example, a water-dispersible pharmaceutical composition for oral administration can comprise:
a) an intragranular portion comprising one or more of a diluent, disintegrant, binder, lubricant and glidant; and
b) an extragranular portion comprising at least one anti-retroviral drug and one or more of a diluent, disintegrant, lubricant and glidant.

In another example, a water-dispersible pharmaceutical composition for oral administration can comprise:
a) an intragranular portion comprising a diluent, disintegrant and optionally a binder; and
b) an extragranular portion comprising lamivudine, stavudine and one or more of a diluent, disintegrant, lubricant and glidant.

In another example, a water-dispersible pharmaceutical composition for oral administration can comprise:
a) an intragranular portion comprising stavudine and/or lamivudine, one or more of a diluent, disintegrant, lubricant and glidant; and
b) an extragranular portion comprising one or more of a diluent, disintegrant, lubricant and glidant.

In another example, a water-dispersible pharmaceutical composition for oral administration can comprise:
a) an intragranular portion comprising stavudine and lamivudine, one or more of a diluent, disintegrant, lubricant and glidant; and
b) an extragranular portion comprising one or more of a diluent, disintegrant, lubricant and glidant.

In another example, a water-dispersible pharmaceutical composition for oral administration can comprise:
a) a first granular portion comprising diluent, disintegrant and optionally a binder;
b) a second granular portion comprising stavudine and one or more of a diluent, lubricant and glidant; and
c) an extragranular portion comprising lamivudine and one or more of a diluent, disintegrant, lubricant and glidant.

In another example, a water-dispersible pharmaceutical composition for oral administration can comprise:
a) an intragranular portion comprising nevirapine, diluent, disintegrant and optionally a binder; and
b) an extragranular portion comprising lamivudine, stavudine, and one or more of a diluent, disintegrant, lubricant and glidant.

In another example, a water-dispersible pharmaceutical composition for oral administration can comprise:
a) a first granular portion comprising nevirapine, diluent, disintegrant and optionally a binder;
b) a second granular portion comprising diluent, disintegrant and optionally a binder; and
c) an extragranular portion comprising lamivudine, stavudine and one or more of a diluent, disintegrant, lubricant and glidant.

In another example, a water-dispersible pharmaceutical composition for oral administration can comprise:
a) a first granular portion comprising nevirapine, diluent, disintegrant and optionally a binder;
b) a second granular portion comprising stavudine and one or more of a diluent, lubricant and glidant; and
c) an extragranular portion comprising lamivudine, lubricant, glidant and optionally a diluent.

In another example, a water-dispersible pharmaceutical composition for oral administration can comprise:
a) a first granular portion comprising nevirapine and one or more of a diluent, lubricant and glidant,
b) a second granular portion comprising stavudine and/or lamivudine, one or more of a diluent, lubricant and glidant; and
c) an extragranular potion comprising one or more of diluent, disintegrant, lubricant and glidant.

The pharmaceutical compositions described herein are meant for oral administration and may be utilized in the form of granules or tablets. More particularly, such pharmaceutical compositions are meant to be dispersed in water, other solvent or other suitable vehicle prior to administration. The described pharmaceutical compositions disperse in water in less than one minute. The dispersion formed is visually uniform with no substantial sedimentation and does not exhibit any feeling of grittiness in the mouth on oral intake.

The pharmaceutical compositions as described herein may include one or more pharmaceutically acceptable excipients. Suitable pharmaceutically acceptable excipients can be selected from diluents, binders, disintegrants, lubricants, glidants or mixtures thereof.

Suitable diluents include saccharides, e.g., lactose, dextrose, sucrose, fructose, maltose or mixtures thereof; sugars, e.g., mannitol, erythritol, sorbitol, xylitol, lactitol or mixtures thereof; cellulose derivatives, e.g., powdered cellulose, microcrystalline cellulose, silicified microcrystalline cellulose, dicalcium phosphate, tribasic calcium phosphate, calcium sulphate, calcium carbonate and the like, or mixtures thereof. Particularly suitable diluents are lactose, microcrystalline cellulose, silicified microcrystalline cellulose or mixtures thereof. The diluent may be present in an amount from about 25% to about 75% by weight of the pharmaceutical composition.

Disintegrants play a major role in the disintegration of compositions as described herein. The disintegrant may be selected from sodium starch glycolate, cross-linked carboxymethylcellulose and its sodium salt, cross-linked polyvinylpyrrolidone, pregelatinised starch, sodium carboxymethyl cellulose, calcium carboxymethyl cellulose, low-substituted hydroxypropyl cellulose, alginates and the like. Particularly suitable disintegrant is sodium starch glycolate. The disintegrant may be present in an amount from about 1% to 20% by weight of the composition.

Binders are generally used in a solid dosage form to impart cohesive properties to a powdered blend. Binders may be a dry binder such as microcrystalline cellulose, which can be particularly useful in a dry granulation process. Binders may also be selected from gums, e.g., acacia, guar gum, alginic acid, sodium alginate or mixtures thereof; starch derivatives, e.g., corn starch, pregelatinised starch; polyvinylpyrrolidone, ethyl cellulose, methyl cellulose, hydroxyethyl cellulose, hydroxypropyl cellulose, hydroxypropyl methylcellulose, carboxyvinyl polymers, e.g., carbomers, acrylates, e.g., Eudragits and other such materials routinely used in the art of solid dosage form manufacturing. Particularly suitable binders include polyvinyl pyrrolidone. The binder may be present in an amount from about 0.5% to about 15% by weight of the pharmaceutical composition.

Lubricants may be selected from talc, magnesium stearate, zinc stearate, calcium stearate, sodium stearyl fumarate, stearic acid or mixtures thereof. Glidants may be selected from talc, colloidal silicon dioxide, and the like, or mixtures thereof. Lubricants and glidants may be used in an amount from about 0.1% to 2% by weight of the pharmaceutical composition.

The water-dispersible pharmaceutical compositions described herein may also include one or more additional excipients, e.g., sweeteners, flavors, colors or mixtures thereof. Sweeteners may be selected from aspartame, saccharine sodium, sucrose, dextrose, fructose, sorbitol and the like, or mixtures thereof.

The water-dispersible pharmaceutical compositions described herein may be prepared by direct compression or by granulation, such as wet or dry granulation or a combination of wet and dry granulation. In direct compression, one or more anti-retroviral drugs may be blended with one or more pharmaceutically acceptable excipients, e.g., diluent, disintegrant, binder, lubricant, glidant or mixtures thereof, and compressed into a tablet. In wet granulation, a blend comprising one or more pharmaceutically acceptable excipients, e.g., diluent and disintegrant, and optionally one or more anti-retroviral drugs, may be granulated with a solution or dispersion of one or more binders. Alternatively, one or more binders may be added to the above blend and the resulting blend granulated with one or more suitable solvents. The resulting granules may be dried, and if needed, sized and subsequently blended with one or more anti-retroviral drugs or granules comprising one or more anti-retroviral drugs, and one or more pharmaceutically acceptable excipients, e.g., diluent, disintegrant, lubricant, glidants or mixtures thereof, and compressed into a tablet.

Dry granulation may be carried out by slugging or roller compaction. Particularly suitable is roller compaction. In some embodiments, one or more anti-retroviral drugs and one or more of pharmaceutical excipients selected from diluent, disintegrant, binder, glidant, lubricant or mixtures thereof can be blended and transferred to a roller compactor and compacted into a sheet. The resulting compact sheet may be fed to a mill, such as an oscillatory mill filled with a screen. After passing through the mill and the screen, the compact can be converted into granules of desired particle size distribution. The granules may further be mixed with one or more anti-retroviral drugs or granules of one or more anti-retroviral drugs, and one or more pharmaceutically acceptable excipients, e.g., diluent, disintegrant, lubricant, glidant or mixtures thereof and compressed into a tablet.

Water-dispersible pharmaceutical compositions can be prepared by a process comprising the steps of:

a. forming a first blend comprising one or more pharmaceutically acceptable excipients selected from one or more diluents, disintegrants, binders, lubricants, glidants or mixtures thereof and one or more anti-retroviral drugs;

b. granulating the first blend by wet or dry granulation to form granules;

c. blending the granules with of one or more pharmaceutically acceptable excipients selected from one or more diluents, disintegrants, binders, lubricants, glidants or mixtures thereof and one or more anti-retroviral drugs to form a second blend; and

d. forming the second blend into a pharmaceutical composition,

wherein the one or more anti-retroviral drugs are selected from lamivudine, zidovudine, stavudine, abacavir, adefovir, tenofovir, emtricitabine, zalcitabine, didanosine, efavirenz, nevirapine, delavirdine, indinavir, nelfinavir, lopinavir, ritonavir, saquinavir, amprenavir, atazanavir, tipranavir, fosamprenavir or mixtures thereof.

In one embodiment, forming the second blend into a pharmaceutical composition comprises compressing the second blend to form a tablet. In another embodiment, forming the first blend comprises forming a first granular portion by blending one or more pharmaceutically acceptable excipients selected from one or more diluents, disintegrants, binders, lubricants, glidants or mixtures thereof and one or more anti-retroviral drugs, forming a second granular portion by blending one or more pharmaceutically acceptable excipients selected from one or more diluents, disintegrants, binders, lubricants, glidants or mixtures thereof and optionally one or more anti-retroviral drugs, and blending the first granular portion with the second granular portion to form a first blend.

Water-dispersible pharmaceutical compositions can also be prepared by a process comprising the steps of:

a. forming a first blend comprising one or more pharmaceutically acceptable excipients selected from one or more diluents, disintegrants, binders, lubricants, glidants or mixtures thereof and one or more anti-retroviral drugs;

b. granulating the first blend by wet or dry granulation to form granules;

c. blending the granules with of one or more pharmaceutically acceptable excipients selected from one or more diluents, disintegrants, binders, lubricants, glidants or mixtures thereof to form a second blend; and

d. forming the second blend into a pharmaceutical composition, wherein the one or more anti-retroviral drugs are selected from lamivudine, zidovudine, stavudine, abacavir, adefovir, tenofovir, emtricitabine, zalcitabine, didanosine, efavirenz, nevirapine, delavirdine, indinavir, nelfinavir, lopinavir, ritonavir, saquinavir, amprenavir, atazanavir, tipranavir, fosamprenavir or mixtures thereof.

In one embodiment, forming the second blend into a pharmaceutical composition comprises compressing the second blend to form a tablet. In another embodiment, forming the first blend comprises forming a first granular portion by blending one or more pharmaceutically acceptable excipients selected from one or more diluents, disintegrants, binders, lubricants, glidants or mixtures thereof and one or more anti-retroviral drugs, forming a second granular portion by blending one or more pharmaceutically acceptable excipients selected from one or more diluents, disintegrants, binders, lubricants, glidants or mixtures thereof and optionally one or more anti-retroviral drugs, and blending the first granular portion with the second granular portion to form a first blend.

Water-dispersible pharmaceutical compositions can also be prepared by a process comprising the steps of:

a. forming a first blend comprising one or more pharmaceutically acceptable excipients selected from one or more diluents, disintegrants, binders, lubricants, glidants or mixtures thereof;

b. granulating the first blend by wet or dry granulation to form granules;

c. blending the granules with of one or more pharmaceutically acceptable excipients selected from one or more diluents, disintegrants, binders, lubricants, glidants or mixtures and one or more anti-retroviral drugs thereof to form a second blend; and

d. forming the second blend into a pharmaceutical composition, wherein the one or more anti-retroviral drugs are selected from lamivudine, zidovudine, stavudine, abacavir, adefovir, tenofovir, emtricitabine, zalcitabine, didanosine, efavirenz, nevirapine, delavirdine, indinavir, nelfinavir, lopinavir, ritonavir, saquinavir, amprenavir, atazanavir, tipranavir, fosamprenavir or mixtures thereof.

In one embodiment, forming the second blend into a pharmaceutical composition comprises compressing the second blend to form a tablet.

Examples of processes of preparing water-dispersible pharmaceutical compositions described herein include, but are not limited to, the following. For example, water-dispersible tablet for oral administration may be prepared by:
preparing a blend of one or more of a diluent, disintegrant, binder, lubricant and optionally an anti-retroviral drug selected from nevirapine, stavudine or lamivudine;
granulating the blend by either wet or dry granulation;
blending the granules with one or more a diluent, disintegrant, binder, lubricant, glidant and at least one anti-retroviral drug selected from nevirapine, stavudine or lamivudine; and
compressing the blend into a tablet

In another example, a water-dispersible tablet for oral administration may be prepared by
preparing a blend of diluent and disintegrant;
granulating the blend with water;
drying the granules;
blending the granules with lamivudine, stavudine, lubricant and glidant; and
compressing the blend into a tablet.

In another example, a water-dispersible tablet for oral administration may be prepared by
preparing a first granular portion by blending a diluent and disintegrant, granulating the blend with water and drying the granules;
preparing a second granular portion by blending stavudine, binder and lubricant, compacting the blend and milling;
blending the granules obtained above with lamivudine, lubricant and glidant; and
compressing the blend into a tablet.

In another example, a water-dispersible tablet for oral administration may be prepared by
preparing a blend of a nevirapine, diluent and disintegrant;
granulating the blend with an aqueous solution of a binder;
drying the granules;
blending the granules with lamivudine, stavudine, diluent, disintegrant, lubricant and glidant; and
compressing the blend into a tablet.

In another example, a water-dispersible tablet for oral administration may be prepared by
preparing a first granular portion by blending nevirapine, diluent and disintegrant, granulating the blend with an aqueous solution of binder and drying the granules;
preparing a second granular portion by blending diluent and disintegrant and granulating the blend with water and drying the granules;
blending the first and second granular portions with lamivudine, stavudine, diluent, lubricant and glidant; and compressing the blend into a tablet.

In another example, a water-dispersible tablet for oral administration may be prepared by
preparing a first granular portion by blending nevirapine, diluent and disintegrant, granulating the blend with an aqueous solution of a binder and drying the granules;
preparing a second granular portion by blending stavudine, diluent, lubricant and glidant, compacting the blend and milling;
blending the granules obtained above with lamivudine, diluent, lubricant and glidant; and
compressing the blend into a tablet.

In another example, a water-dispersible tablet for oral administration may be prepared by
preparing a first granular portion by blending nevirapine, diluent and disintegrant, granulating the blend with water and drying the granules;
preparing a second granular portion by blending stavudine, diluent, lubricant and glidant, compacting the blend and milling;
blending the granules obtained above with lamivudine, diluent, disintegrant, lubricant and glidant; and
compressing the blend into a tablet.

In another example, a water-dispersible tablet form oral administration may be prepared by
preparing a first granular portion by blending nevirapine and a lubricant, compacting the blend and milling,
preparing a second granular portion by blending stavudine, lamivudine, diluent, lubricant and glidant, compacting the blend and milling;
blending the granules obtained above with one or more of diluent, disintegrant, lubricant and glidant; and
compressing the blend into a tablet.

In another example, a water-dispersible tablet form oral administration may be prepared by
preparing a granular portion by blending stavudine, lamivudine, diluent, lubricant and glidant, compacting the blend and milling;
blending the granules obtained above with one or more of diluent, disintegrant, lubricant and glidant; and
compressing the blend into a tablet.

The pharmaceutical compositions as described herein may further be illustrated by the following examples but these should not be construed as limiting the scope of the invention:

### EXAMPLES

### Example 1

| Ingredients | Amount (in mg) |
|---|---|
| Intragranular | |
| Nevirapine | 70.0 |
| Lactose monohydrate | 70.0 |
| Polyvinyl pyrrolidone | 6.0 |
| Microcrystalline cellulose | 141.4 |
| Sodium starch glycolate | 25.0 |
| Purified water | q.s |
| Extragranular | |
| Lamivudine | 40.0 |
| Stavudine | 10.0 |
| Microcrystalline cellulose | 20.0 |
| Colloidal silicon dioxide | 2.0 |
| Aspartame | 8.0 |
| Flavor | 3.6 |
| Magnesium stearate | 4.0 |
| Total weight | 400.0 |

A first granular portion was prepared by granulating a blend of nevirapine and lactose with an aqueous solution of polyvinyl pyrrolidone. A second granular portion was prepared by granulating a blend of microcrystalline cellulose and sodium starch glycolate with water. The first and the second granular portions were subsequently blended with lamivudine and stavudine. The blend obtained was mixed with microcrystalline cellulose, colloidal silicon dioxide, aspartame, flavor and magnesium stearate; and was compressed into a tablet using appropriate tooling.

### Example 2

| Ingredients | Amount (in mg) |
|---|---|
| Intragranular | |
| Nevirapine | 70.0 |
| Lactose monohydrate | 70.0 |
| Polyvinyl pyrrolidone | 6.0 |
| Sodium starch glycolate | 12.0 |
| Purified water | q.s |
| Extragranular | |
| Lamivudine | 40.0 |
| Stavudine | 10.0 |
| Silicified microcrystalline cellulose | 166.4 |
| Sodium starch glycolate | 8.0 |
| Colloidal silicon dioxide | 2.0 |
| Aspartame | 8.0 |
| Flavor | 3.6 |
| Magnesium stearate | 4.0 |
| Total weight | 400.0 |

Nevirapine, lactose and sodium starch glycolate were blended and subsequently granulated with an aqueous solution of polyvinyl pyrrolidone. The granules were blended with lamivudine and stavudine. The blend obtained was mixed with silicified microcrystalline cellulose, sodium starch glycolate, colloidal silicon dioxide, aspartame, flavor and magnesium stearate; and was compressed into a tablet using appropriate tooling.

### Example 3

| Ingredients | Amount (in mg) |
|---|---|
| Intragranular | |
| Nevirapine | 70.0 |
| Stavudine | 10.0 |
| Microcrystalline cellulose | 192.27 |
| Sodium starch glycolate | 40.0 |
| Colloidal silicon dioxide | 0.6 |
| Magnesium stearate | 0.5 |
| Purified water | q.s |
| Extragranular | |
| Lamivudine | 40.0 |
| Microcrystalline cellulose | 32.23 |
| Colloidal silicon dioxide | 1.4 |
| Aspartame | 8.0 |
| Flavor | 1.5 |
| Magnesium stearate | 3.5 |
| Total weight | 400.0 |

A first granular portion was prepared by granulating a blend of nevirapine, microcrystalline cellulose and sodium starch glycolate with purified water, and drying the granules. A second granular portion was prepared by blending stavudine, microcrystalline cellulose, colloidal silicon dioxide and magnesium stearate, compacting the blend in a roller compactor and milling the compacts. The first and the second granular portions were subsequently blended with lamivudine, microcrystalline cellulose, colloidal silicon dioxide, aspartame, flavor and magnesium stearate, and compressed into a tablet using appropriate tooling.

### Example 4

| Ingredients | Amount (in mg) |
|---|---|
| Intragranular | |
| Nevirapine | 70.0 |
| Stavudine | 10.0 |
| Lactose monohydrate | 70.79 |
| Microcrystalline cellulose | 36.10 |
| Sodium starch glycolate | 12.0 |
| Polyvinyl pyrrolidone | 6.0 |
| Colloidal silicon dioxide | 0.6 |
| Magnesium stearate | 0.5 |
| Purified water | q.s |
| Extragranular | |
| Lamivudine | 40.0 |
| Silicified microcrystalline | 131.61 |
| cellulose | |
| Sodium starch glycolate | 8.0 |
| Colloidal silicon dioxide | 1.4 |
| Aspartame | 8.0 |
| Flavor | 1.5 |
| Magnesium stearate | 3.5 |
| Total weight | 400.0 |

A first granular portion was prepared by granulating a blend of nevirapine, lactose and sodium starch glycolate with an aqueous solution of polyvinyl pyrrolidone, and drying the granules. A second granular portion was prepared by blending stavudine, microcrystalline cellulose, colloidal silicon dioxide and magnesium stearate, compacting the blend in a roller compactor and milling the compacts. The first and the second granular portions were subsequently blended with lamivudine, silicified microcrystalline cellulose, sodium starch glycolate, colloidal silicon dioxide, aspartame, flavor and magnesium stearate, and compressed into a tablet using appropriate tooling.

### Example 5

| Ingredients | Amount (in mg) |
|---|---|
| Intragranular | |
| Stavudine | 10.0 |
| Microcrystalline cellulose | 140.3 |
| Sodium starch glycolate | 50.0 |
| Magnesium stearate | 0.5 |
| Purified water | q.s. |
| Extragranular | |
| Lamivudine | 40.0 |
| Colloidal silicon dioxide | 1.25 |
| Flavor | 0.95 |
| Aspartame | 5.0 |
| Magnesium stearate | 2.0 |
| Total weight | 250.0 |

A first granular portion was prepared by granulating a blend of microcrystalline cellulose and sodium starch glycolate with purified water, and drying the granules. A second granular portion was prepared by blending stavudine, microcrystalline cellulose and magnesium stearate, compacting the blend in a roller compactor and milling the compacts. The first and the second granular portions were subsequently blended with lamivudine, colloidal silicon dioxide, aspartame, flavor and magnesium stearate, and compressed into a tablet using appropriate tooling.

### Example 6

| Ingredients | Amount (in mg) |
|---|---|
| Intragranular | |
| Microcrystalline cellulose | 139.75 |
| Sodium starch glycollate | 50.0 |
| Purified water | q.s |
| Extragranular | |
| Lamivudine | 40.0 |
| Stavudine | 10.0 |
| Colloidal silicon dioxide | 1.25 |
| Flavor | 1.5 |
| Aspartame | 5.0 |
| Magnesium stearate | 2.5 |
| Total weight | 250.0 |

Microcrystalline cellulose and sodium starch glycolate were blended and granulated with water. The granules were blended with lamivudine and stavudine. The blend obtained was mixed with colloidal silicon dioxide, aspartame, flavor and magnesium stearate; and was compressed into a tablet using appropriate tooling.

The water-dispersibility of the pharmaceutical compositions as described herein may be determined by evaluation of disintegration time. Thus, the tablets prepared as described in Examples 1 to 6 were evaluated for their disintegration time using conventional apparatus such as a USP disintegration apparatus. In such a procedure, the tablets are placed in water maintained at 37° C. wherein the motion of the disintegration apparatus simulates the passage of a tablet through the body and the time required for each tablet to break down is noted. The disintegration time of tablets of Example 1 to 6 is given in Table 1.

TABLE 1
Disintegration time of tablets of Examples 1 to 6:

| | Disintegration time* |
|---|---|
| | (in seconds) |
| Example 1 | 40 |
| Example 2 | 40 |
| Example 3 | 30 |
| Example 4 | 45 |
| Example 5 | 20 |
| Example 6 | 20 |

*as measured in a disintegration apparatus

Thus, the tablets as described in Examples 1 to 6 may be placed in 10 ml of water wherein the tablet will disintegrate and disperse within the time as described in Table 1 to give a visually uniform dispersion.

### Example 7

| Ingredients | Amount (in mg) |
|---|---|
| Intragranular | |
| Microcrystalline cellulose | 139.75 |
| Sodium starch glycollate | 50.0 |
| Purified water | q.s |
| Extragranular | |
| Lamivudine | 40.0 |
| Colloidal silicon dioxide | 1.25 |
| Flavor | 1.5 |
| Aspartame | 5.0 |
| Magnesium stearate | 2.5 |
| Total weight | 240.0 |

Microcrystalline cellulose and sodium starch glycolate were blended and granulated with water. The granules were blended with lamivudine, colloidal silicon dioxide, aspartame, flavor and magnesium stearate; and was compressed into a tablet using appropriate tooling.

### Example 8

| Ingredients | Amount (in mg) |
|---|---|
| Intragranular-I | |
| Nevirapine | 70.0 |
| Magnesium stearate | 1.0 |
| Intragranular-II | |
| Stavudine | 10.0 |
| Lamivudine | 40.0 |
| Microcrystalline cellulose | 60.0 |
| Colloidal silicon dioxide | 2.0 |
| Magnesium stearate | 1.0 |
| Extragranular | |
| Microcrystalline cellulose | 180.27 |
| Sodium starch glycolate | 20.0 |
| Colloidal silicon dioxide | 2.0 |
| Aspartame | 8.0 |
| Flavor | 1.5 |
| Magnesium stearate | 2.0 |
| Total weight | 400.0 |

Nevirapine and magnesium stearate were blended and compacted on a roller compactor. The compacts were milled into granules. Stavudine, lamivudine, colloidal silicon dioxide and microcrystalline cellulose were mixed and lubricated with magnesium stearate and compacted on a roller compactor. The compacts were milled into granules. The two granular portions were mixed with extragranular microcrystalline cellulose, sodium starch glycolate, aspartame, colloidal silicon dioxide, flavor and magnesium stearate and compressed into a tablet using appropriate tooling.

### Example 9

| Ingredients | Amount (in mg) |
|---|---|
| Intragranular | |
| Stavudine | 10.0 |
| Lamivudine | 40.0 |
| Microcrystalline cellulose | 60.0 |
| Colloidal silicon dioxide | 0.5 |
| Magnesium stearate | 0.5 |
| Extragranular | |
| Microcrystalline cellulose | 117.80 |
| Sodium starch glycolate | 12.5 |
| Colloidal silicon dioxide | 0.75 |
| Aspartame | 5.0 |
| Flavor | 0.95 |
| Magnesium stearate | 2.0 |
| Total weight | 250.0 |

Stavudine, lamivudine, colloidal silicon dioxide and microcrystalline cellulose were mixed and lubricated with magnesium stearate and compacted on a roller compactor. The compacts were milled into granules. The granules were mixed with extragranular microcrystalline cellulose, sodium starch glycolate, aspartame, colloidal silicon dioxide, flavor and magnesium stearate and compressed into a tablet using appropriate tooling.

### Example 10

In an open label, balanced, randomized, two-treatment, two sequence, two period, 15 single dose, crossover, bioavailability study comparing the dispersible tablets of Example 8 (Test) containing lamivudine 40 mg, nevirapine 70 mg and stavudine 10 mg were compared with 4 mL of Epivir® oral solution (containing lamivudine 10 mg/mL) of GlaxoSmithkline, 7 mL of Viramune® oral suspension (containing nevirapine 50 mg/5 mL) of Boehringer Ingelheim Pharmaceuticals Inc. and 10 mL of Zerit® oral solution (containing stavudine 1 mg/mL) of Bristol-Myers Squibb (as References) in healthy seventeen adult male human subjects under fasting condition. The assessed pK parameters were Tₘₐₓ, Cₘₐₓ, AUC₀₋ₜ, and AUC_{0-∞}. The pK parameters of test and references are given in Table 2.

TABLE 2
Summary statistics of pharmacokinetic parameters (mean) of lamivudine,
nevirapine and stavudine for Tests and References in 17 healthy
adult male human subjects under fasting condition.

| | | | | |
|---|---|---|---|---|
| Product/ | Cₘₐₓ | AUC₀₋ₜ | AUC_{0-∞} | |
| Statistics | (ng/mL) | (ngh/mL) | (ngh/mL) | Tₘₐₓ |

Lamivudine

| | | | | |
|---|---|---|---|---|
| Reference | 365.447 | 1434.311 | 1511.855 | 0.796 |
| (Epivir ®) | | | | |
| Test | 399.241 | 1432.633 | 1494.623 | 0.829 |
| T/R ratio | 108.58% | 100.86% | 99.79% | — |
| 90% | 94.99-124.10% | 93.02-109.36% | 91.76-108.53% | — |
| Confidence | | | | |
| interval | | | | |

Nevirapine

| | | | | |
|---|---|---|---|---|
| Reference | 992.282 | 57867.709 | 65839.154 | 3.868 |
| Viramune ® | | | | |
| Test | 893.306 | 53001.657 | 60046.966 | 1.824 |
| T/R ratio | 89.62% | 91.74% | 91.50% | — |
| 90% | 81.02-99.12% | 83.14-101.24% | 82.12-101.96% | — |
| Confidence | | | | |
| interval | | | | |

Stavudine

| | | | | |
|---|---|---|---|---|
| Reference | 242.164 | 410.677 | 431.735 | 0.370 |
| Zerit ® | | | | |
| Test | 250.054 | 434.177 | 454.049 | 0.430 |
| T/R ratio | 103.07% | 104.94% | 104.47% | — |
| 90% | 94.34-112.61% | 99.54-110.63% | 99.45-109.74% | — |
| Confidence | | | | |
| interval | | | | |

Test product (Example 8), containing lamivudine 40 mg, nevirapine 70 mg and stavudine 10 mg dispersible tablets of the invention was bioequivalent to Epivir® oral solution (containing lamivudine 10 mg/mL) of GlaxoSmithKline, Viramune® oral suspension (containing nevirapine 50 mg/5 mL) of Boehringer Ingelheim Pharmaceuticals Inc. and Zerit® oral solution (containing stavudine 1 mg/mL) of Bristol-Myers Squibb in the present study under fasting conditions. The 90% confidence intervals for Cmax, AUC₀₋ₜ and AUC_{0-∞} were within the range of 80-125%.

### Example 11

In an open label, balanced, randomized, two-treatment, two sequence, two period, single dose, crossover, bioavailability study comparing the dispersible tablets of Example 8 (Test) containing lamivudine 40 mg, nevirapine 70 mg and stavudine 10 mg were compared with 4 mL of Epivir® oral solution (containing lamivudine 10 mg/mL) of Glaxosmithkline, 7 mL of Viramune® oral suspension (containing nevirapine 50 mg/5 mL) of Boehringer Ingelheim Pharmaceuticals Inc. and 10 ml of Zerit® oral solution (containing stavudine 1 mg/mL) of Bristol-Myers Squibb (as References) in healthy seventeen adult male human subjects under fed condition. The assessed pK parameters were Tₘₐₓ, Cₘₐₓ, AUC₀₋ₜ, and AUC_{0-∞}. The pK parameters of test and references are given in Table 3.

TABLE 3
Summary statistics of pharmacokinetic parameters (mean) of lamivudine,
nevirapine and stavudine for Tests and References in 17 healthy
adult male human subjects under fed condition.

| | | | | |
|---|---|---|---|---|
| Product/ | Cₘₐₓ | AUC₀₋ₜ | AUC_{0-∞} | |
| Statistics | (ng/mL) | (ngh/mL) | (ngh/mL) | Tₘₐₓ |

Lamivudine

| | | | | |
|---|---|---|---|---|
| Reference | 213.464 | 1535.520 | 1706.746 | 2.000 |
| (Epivir ®) | | | | |
| Test | 232.299 | 1571.984 | 1766.974 | 2.484 |
| T/R ratio | 106.71% | 103.53% | 104.81% | — |
| 90% | 94.51-120.48% | 95.76-111.94% | 95.68-114.82% | — |
| Confidence | | | | |
| interval | | | | |

Nevirapine

| | | | | |
|---|---|---|---|---|
| Reference | 965.562 | 71927.683 | 90059.965 | 5.969 |
| Viramune ® | | | | |
| Test | 987.206 | 67991.947 | 91125.454 | 9.688 |
| T/R ratio | 102.08% | 93.73% | 102.00% | — |
| 90% | 96.09-108.45% | 85.37-102.91% | 96.61-107.69% | — |
| Confidence | | | | |
| interval | | | | |

Stavudine

| | | | | |
|---|---|---|---|---|
| Reference | 125.205 | 473.432 | 505.997 | 1.114 |
| Zerit ® | | | | |
| Test | 137.555 | 439.587 | 463.380 | 1.635 |
| T/R ratio | 105.79% | 93.02% | 92.04% | — |
| 90% | 94.18-118.83% | 88.03-98.31% | 87.08-97.29% | — |
| Confidence | | | | |
| interval | | | | |

Test product (Example 8), containing lamivudine 40 mg, nevirapine 70 mg and stavudine 10 mg dispersible tablets of the invention was bioequivalent to Epivir® oral solution (containing lamivudine 10 mg/mL) of Glaxosmithkline, Viramune® oral suspension (containing nevirapine 50 mg/5 mL) of Boehringer Ingelheim Pharmaceuticals Inc. and Zerit® oral solution (containing stavudine 1 mg/mL) of Bristol-Myers Squibb in the present study under fed conditions. The 90% confidence intervals for Cₘₐₓ, AUC₀₋ₜ, and AUC_{0-∞} were within the range of 80-125%.

### Example 12

In an open label, balanced, randomized, two-treatment, two-period, two-sequence, single-dose, crossover bioavailability study comparing dispersible tablets of Example 8 with 4 mL of Epivir® oral solution (containing lamivudine 10 mg/mL), Viramune® suspension (containing 50 mg/5 mL) and 10 ml of Zerit® oral solution (containing stavudine 1 mg/mL) in 36 healthy adult male human subjects under fasting condition. Out of 36 subjects, 32 subjects completed all the periods of the crossover study.

TABLE 4
Summary statistics of pharmacokinetic parameters (mean) of lamivudine,
nevirapine and stavudine for Tests and References in 32 healthy
adult male human subjects under fasting condition.

| | | | |
|---|---|---|---|
| Product/ | Cₘₐₓ | AUC₀₋ₜ | AUC_{0-∞} |
| Statistics | (ng/mL) | (ngh/mL) | (ngh/mL) |

Lamivudine

| | | | |
|---|---|---|---|
| Reference | 496.321 | 2137.128 | 2241.227 |
| (Epivir ®) | | | |
| Test | 573.628 | 2300.209 | 2397.625 |
| T/R ratio | 115.27% | 107.90% | 107.24% |
| 90% | 106.84-124.38% | 100.87-115.41% | 100.54-114.39% |
| Confidence | | | |
| interval | | | |

Nevirapine

| | | | |
|---|---|---|---|
| Reference | 1162.80 | 69074.642 | 76442.788 |
| Viramune ® | | | |
| Test | 1219.45 | 68659.468 | 76947.178 |
| T/R ratio | 105.11% | 99.56 | 100.84 |
| 90% | 98.90-111.71% | 95.59-103.69% | 96.77-105.08% |
| Confidence | | | |
| interval | | | |

Stavudine

| | | | |
|---|---|---|---|
| Reference | 382.321 | 616.451 | 651.470 |
| Zerit ® | | | |
| Test | 339.036 | 565.618 | 605.272 |
| T/R ratio | 88.73% | 91.87% | 92.82% |
| 90% | 83.42-94.39% | 89.24-94.58% | 90.95-94.73% |
| Confidence | | | |
| interval | | | |

Test product (Example 8), containing lamivudine 40 mg, nevirapine 70 mg and stavudine 10 mg was bioequivalent to Epivir® oral solution (containing lamivudine 10 mg/mL), Viramune® oral suspension (containing nevirapine 50 mg/5 mL) and Zerit® oral solution (containing stavudine 1 mg/mL) in the present study under fasting conditions. The 90% confidence intervals for Cₘₐₓ, AUC₀₋ₜ and AUC_{0-∞} were within the range of 80-125%.

While several particular forms of the inventions have been described, it will be apparent that various modifications and combinations of the inventions detailed in the text can be made without departing from the spirit and scope of the inventions. Accordingly, it is not intended that the inventions be limited, except as by the appended claims.

We claim:

1. A water-dispersible pharmaceutical compositions for oral administration comprising one or more anti-retroviral drugs and one or more pharmaceutically acceptable excipients selected from one or more diluents, disintegrants, binders, lubricants, glidants or mixtures thereof.

2. The water-dispersible pharmaceutical composition of claim 1 comprising a combination of at least two anti-retroviral drugs.

3. The water-dispersible pharmaceutical composition of claim 2, wherein the composition comprises:
a) an intragranular portion comprising pharmaceutically acceptable excipients selected from one or more diluents, disintegrants, binders, lubricants, glidants or mixtures thereof and one or more anti-retroviral drugs; and
b) an extragranular portion comprising pharmaceutically acceptable excipients selected from one or more diluents, disintegrants, lubricants, glidants or mixtures thereof and one or more anti-retroviral drugs.

4. The water-dispersible pharmaceutical composition of claim 2, wherein the composition comprises:
a) an intragranular portion comprising pharmaceutically acceptable excipients selected from one or more diluents, disintegrants, binders, lubricants, glidants or mixtures thereof and one or more anti-retroviral drugs; and
b) an extragranular portion comprising pharmaceutically acceptable excipients selected from one or more diluents, disintegrants, lubricants, glidants or mixtures thereof.

5. The water-dispersible pharmaceutical composition of claim 2, wherein the composition comprises:
a) an intragranular portion comprising pharmaceutically acceptable excipients selected from one or more diluents, disintegrants, binders, lubricants, glidants or mixtures thereof; and
b) an extragranular portion comprising pharmaceutically acceptable excipients selected from one or more diluents, disintegrants, lubricants, glidants or mixtures thereof.

6. The water-dispersible pharmaceutical composition of claim 1, wherein the one or more anti-retroviral drugs are selected from lamivudine, zidovudine, stavudine, abacavir, adefovir, tenofovir, emtricitabine, zalcitabine, didanosine, efavirenz, nevirapine, delavirdine, indinavir, nelfinavir, lopinavir, ritonavir, saquinavir, amprenavir, atazanavir, tipranavir, fosamprenavir or mixtures thereof.

7. The water-dispersible pharmaceutical composition of claim 1, wherein the pharmaceutical composition disintegrates and disperses in one or more solvents or a vehicle of administration in less than one minute.

8. The water-dispersible pharmaceutical composition of claim 1, wherein the disintegrants are selected from sodium starch glycolate, cross-linked carboxymethylcellulose and its sodium salt, cross-linked polyvinylpyrrolidone, pregelatinised starch, sodium carboxymethyl cellulose, calcium carboxymethyl cellulose, low-substituted hydroxypropyl cellulose, alginates or its salts or mixtures thereof.

9. The water-dispersible pharmaceutical composition of claim 1, wherein the diluents are selected from lactose, dextrose, sucrose, fructose, maltose, powdered cellulose, microcrystalline cellulose, mannitol, erythritol, sorbitol, xylitol lactitol, dicalcium phosphate, tribasic calcium phosphate, calcium sulphate, calcium carbonate or mixtures thereof.

10. The water-dispersible pharmaceutical composition of claim 1, wherein the binders are selected from corn starch, pregelatinised starch, polyvinylpyrrolidone, hydroxypropyl cellulose, hydroxypropyl methylcellulose, carboxyvinyl polymers, acrylates or mixtures thereof.

11. The water-dispersible pharmaceutical composition of claim 1, wherein the lubricants are selected from talc, magnesium stearate, zinc stearate, calcium stearate, sodium stearyl fumarate, stearic acid or mixtures thereof.

12. The water-dispersible pharmaceutical composition of claim 1, wherein the glidants are selected from talc, colloidal silicon dioxide or mixtures thereof.

13. The water-dispersible pharmaceutical composition of claim 1, wherein the pharmaceutical composition is a tablet.

14. A process for preparing a water-dispersible pharmaceutical composition for oral administration comprising the steps of:
a. forming a first blend comprising one or more pharmaceutically acceptable excipients selected from one or more diluents, disintegrants, binders, lubricants, glidants or mixtures thereof and one or more anti-retroviral drugs;
b. granulating the first blend by wet or dry granulation to form granules;
c. blending the granules with of one or more pharmaceutically acceptable excipients selected from one or more diluents, disintegrants, binders, lubricants, glidants or mixtures thereof and one or more anti-retroviral drugs to form a second blend; and
d. forming the second blend into a pharmaceutical composition,

15. A process for preparing a water-dispersible pharmaceutical composition for oral administration comprising the steps of:
a. forming a first blend comprising one or more pharmaceutically acceptable excipients selected from one or more diluents, disintegrants, binders, lubricants, glidants or mixtures thereof and one or more anti-retroviral drugs;
b. granulating the first blend by wet or dry granulation to form granules;
c. blending the granules with of one or more pharmaceutically acceptable excipients selected from one or more diluents, disintegrants, binders, lubricants, glidants or mixtures thereof to form a second blend; and
d. forming the second blend into a pharmaceutical composition.

16. A process for preparing a water-dispersible pharmaceutical composition for oral administration comprising the steps of:
a. forming a first blend comprising one or more pharmaceutically acceptable excipients selected from one or more diluents, disintegrants, binders, lubricants, glidants or mixtures thereof;
b. granulating the first blend by wet or dry granulation to form granules;
c. blending the granules with of one or more pharmaceutically acceptable excipients selected from one or more diluents, disintegrants, binders, lubricants, glidants or mixtures and one or more anti-retroviral drugs thereof to form a second blend; and
d. forming the second blend into a pharmaceutical composition.
